(19) United States
(12) Patent Application Publication
(10) Pub. No.: US 2024/0226879 A1
(43) Pub. Date: Jul. 11, 2024

(54) DETECTION CHIP AND PREPARATION METHOD THEREOF

(71) Applicants: BEIJING BOE TECHNOLOGY DEVELOPMENT CO., LTD., Beijing (CN); BOE TECHNOLOGY GROUP CO., LTD., Beijing (CN)

(72) Inventors: Zijian ZHAO, Beijing (CN); Houqian GAO, Beijing (CN); Hairong JING, Beijing (CN); Ding DING, Beijing (CN); Shinying LAU, Beijing (CN)

(21) Appl. No.: 17/928,342

(22) PCT Filed: Oct. 29, 2021
(86) PCT No.: PCT/CN2021/127599

§ 371 Date: Nov. 29, 2022

(51) Int. Cl. B01L 3/00

(52) U.S. Cl. CPC B01L 3/502715; B01L 3/502707; B01L 2200/027; B01L 2200/12; B01L 2300/041; B01L 2300/12; B01L 2300/161

(57) ABSTRACT

A detection chip and a preparation method thereof, the detection chip may include a base substrate and a detection layer, the detection layer is provided on the base substrate and include a plurality of detection holes, each of at least a portion of the plurality of detection holes has a hole wall provided with a hydrophilic layer, and a contact angle of the hydrophilic layer is within 20 degrees. The detection chip can implement high-throughput, crosstalk does not easily occur between adjacent detection holes, which, thus, may improve detection accuracy.

### TECHNICAL FIELD

Embodiments of the present disclosure relate to a detection chip and a preparation method thereof.

### BACKGROUND

The DNA sequencing technology is one of the most commonly used technical means in molecular biology-related research, which has promoted rapid development of the field to a certain extent. At present, a sequencing chip may be used to complete a sequencing reaction and a detection process. During the process, structures and the number of independent separating units formed in the sequencing chip directly affect a sequencing effect.

### SUMMARY

At least one embodiment of the present disclosure provides a detection chip, the detection chip comprises a base substrate and a detection layer, the detection layer is provided on the base substrate and includes a plurality of detection holes, each of at least a portion of the plurality of detection holes has a hole wall provided with a hydrophilic layer, and a contact angle of the hydrophilic layer is within 20 degrees.

For example, in the detection chip provided by at least one embodiment of the present disclosure, a contact angle of a surface of the detection layer that is away from the base substrate is 80 degrees to 150 degrees.

For example, in the detection chip provided by at least one embodiment of the present disclosure, slope angles formed by side walls of at least a portion of the plurality of detection holes and a plate surface of the base substrate are 85 degrees to 90 degrees.

For example, in the detection chip provided by at least one embodiment of the present disclosure, a diameter of the detection hole is 0.75 micrometers to 1.75 micrometers; and a separation distance between adjacent detection holes is 0.25 micrometers to 1.25 micrometers.

For example, in the detection chip provided by at least one embodiment of the present disclosure, a depth of the detection hole is 0.75 micrometers to 1.75 micrometers.

For example, in the detection chip provided by at least one embodiment of the present disclosure, a bottom surface of the detection hole that is close to the base substrate comprises an arc surface that is concave toward a direction of the base substrate.

For example, in the detection chip provided by at least one embodiment of the present disclosure, an adapter primer is disposed inside of the detection hole; and the adapter primer is connected to a surface of the hydrophilic layer through a covalent bond.

For example, the detection chip provided by at least one embodiment of the present disclosure further comprises a cover layer, and the cover layer is provided on a side of the adapter primer that is away from the base substrate.

For example, in the detection chip provided by at least one embodiment of the present disclosure, a material of the cover layer comprises a water-soluble polymer.

For example, in the detection chip provided by at least one embodiment of the present disclosure, the water-soluble polymer comprises a copolymer of N-(5-azidoacetamidopentyl)acrylamide and acrylamide.

For example, in the detection chip provided by at least one embodiment of the present disclosure, a material of the detection layer comprises silicon nitride; and a material of the hydrophilic layer comprises silicon oxide.

For example, in the detection chip provided by at least one embodiment of the present disclosure, the detection layer comprises a binding material layer and a hydrophobic material layer; the binding material layer comprises a plurality of holes; and the hydrophobic material layer is provided on the binding material layer, to form a plurality of detection holes at positions of the plurality of holes.

For example, in the detection chip provided by at least one embodiment of the present disclosure, a material of the binding material layer comprises optical clear adhesive; and a material of the hydrophobic material layer comprises silicon nitride.

For example, the detection chip provided by at least one embodiment of the present disclosure further comprises a cover plate, the cover plate is bonded by a binder to a side of the detection layer that is away from the base substrate and comprises a sample inlet and a sample outlet, and the sample inlet and the sample outlet are arranged in an edge region of the cover plate.

For example, in the detection chip provided by at least one embodiment of the present disclosure, the cover plate comprises a plate surface opposite to the base substrate; and a distance between the plate surface and the detection layer is 50 micrometers to 100 micrometers.

For example, in the detection chip provided by at least one embodiment of the present disclosure, the plurality of detection holes are divided into a plurality of groups of detection holes; the plurality of groups of detection holes are arranged in an array; and each of the plurality of groups of detection holes comprises a plurality of detection holes arranged in an array, a separation distance between adjacent two groups of the plurality of groups of detection holes is greater than a separation distance between two adjacent detection holes of the plurality of detection holes in each of the plurality of groups of detection holes.

At least one embodiment of the present disclosure provides a preparation method of a detection chip, the method comprises: providing a base substrate, and forming a detection layer on the base substrate, the detection layer comprising a plurality of detection holes, each of at least a portion of the plurality of detection holes has a hole wall provided with a hydrophilic layer; and a contact angle of the hydrophilic layer is within 20 degrees.

For example, in the preparation method provided by at least one embodiment of the present disclosure, the forming a detection layer on the base substrate comprises: sequentially forming a binding material layer and a metal material layer on the base substrate, patterning the binding material layer and the metal material layer, so that a plurality of holes are formed in the binding material layer and the metal material layer, removing the metal material layer, and forming a hydrophobic material layer on a side of the binding material layer that is away from the base substrate, and the detection layer comprises the binding material layer and the hydrophobic material layer; and the plurality of detection holes are correspondingly formed at positions of the plurality of holes.

For example, in the preparation method provided by at least one embodiment of the present disclosure, the forming a detection layer on the base substrate comprises: sequentially forming a binding material layer and a hydrophobic material layer on the base substrate; and at least patterning the hydrophobic material layer, to form the plurality of detection holes at least in the hydrophobic material layer.

For example, the preparation method provided by at least one embodiment of the present disclosure further comprises forming a hydrophilic layer on a hole wall of the detection hole.

For example, in the preparation method provided by at least one embodiment of the present disclosure, the forming a hydrophilic layer on a hole wall of the detection hole comprises: forming a hydrophilic material layer on a side of the hydrophobic material layer that is away from the base substrate, patterning the hydrophilic material layer, to retain a portion of the hydrophilic material layer that is inside the plurality of detection holes; and removing a portion of the hydrophilic material layer that is between adjacent detection holes of the plurality of detection holes.

For example, the preparation method provided by at least one embodiment of the present disclosure further comprises performing a first surface treatment on the hydrophilic layer and the exposed hydrophobic material layer, so that a contact angle of the hydrophilic layer is within 20 degrees, and a contact angle of the hydrophobic material layer is 80 degrees to 150 degrees.

For example, the preparation method provided by at least one embodiment of the present disclosure further comprises performing a second surface treatment on the hydrophilic layer, so that a surface of the hydrophilic layer has a plurality of activating groups.

For example, in the preparation method provided by at least one embodiment of the present disclosure, the second surface treatment is performed with at least one of GPTMS, 12-mercaptododecanoic acid, and EDC:NHS.

For example, the preparation method provided by at least one embodiment of the present disclosure further comprises connecting adapter primers within the plurality of detection holes, and the adapter primers form covalent bonds with a plurality of activating groups on the surface of the hydrophilic layer, to connect the adapter primers to the surface of the hydrophilic layer through the covalent bonds.

For example, the preparation method provided by at least one embodiment of the present disclosure further comprises forming a cover layer within the plurality of detection holes.

For example, in the preparation method provided by at least one embodiment of the present disclosure, the forming a cover layer within the plurality of detection holes, comprises: forming a cover material layer on a side of the detection layer that is away from the base substrate, performing an ashing process or a polishing process on a portion of the cover material layer that is located between the plurality of detection holes, to remove the portion of the cover material layer that is located between the plurality of detection holes.

For example, in the preparation method provided by at least one embodiment of the present disclosure, the cover material layer comprises a copolymer of N-(5-azidoacetamidopentyl)acrylamide and acrylamide; and the forming a cover material layer on a side of the detection layer that is away from the base substrate, comprises: pre-polymerizing N-(5-azidoacetamidopentyl)acrylamide and acrylamide at a temperature of 40 degrees Celsius to 60 degrees Celsius for 3 minutes to 8 minutes; and coating a pre-polymerized product of N-(5-azidoacetamidopentyl)acrylamide and acrylamide on the side of the detection layer that is away from the base substrate and polymerizing at a temperature of 30 degrees Celsius to 40 degrees Celsius for 1 hour to 3 hours.

For example, the preparation method provided by at least one embodiment of the present disclosure further comprises: coating a binder on a side of the detection layer that is away from the base substrate and on a periphery of the detection layer, covering a cover plate on a side of the binder that is away from the base substrate, pre-baking the detection chip at a temperature of 90 degrees Celsius to 110 degrees Celsius for 3 minutes to 6 minutes, and baking the detection chip at a temperature of 140 degrees Celsius to 160 degrees Celsius for 8 minutes to 12 minutes.

### BRIEF DESCRIPTION OF THE DRAWINGS

In order to more clearly explain the technical scheme of the embodiments of the present disclosure, the following will briefly introduce the drawings of the embodiments. Obviously, the drawings in the following description only relate to some embodiments of the present disclosure, but are not restrictive to the present disclosure.

FIG. 1 is a schematic plan view of a detection chip provided by at least one embodiment of the present disclosure;

FIG. 2 is a cross-sectional schematic diagram of the detection chip in FIG. 1 along an M-M line;

FIG. 3 is another cross-sectional schematic diagram of the detection chip in FIG. 1 along the M-M line;

FIG. 4 is still another cross-sectional schematic diagram of the detection chip in FIG. 1 along the M-M line;

FIG. 5 is yet another cross-sectional schematic diagram of the detection chip in FIG. 1 along the M-M line;

FIG. 6 is another schematic plan view of a detection chip provided by at least one embodiment of the present disclosure;

FIG. 7 is an exploded schematic diagram of a detection chip provided by at least one embodiment of the present disclosure;

FIG. 8 is a schematic diagram of a validity test result of an adapter primer of a detection chip provided by at least one embodiment of the present disclosure; and

FIG. 9A to FIG. 9C, FIG. 10A to FIG. 10B, FIG. 11A to FIG. 11B, FIG. 12, FIG. 13A to FIG. 13B and FIG. 14 are cross-sectional schematic diagrams of a detection chip provided by at least one embodiment of the present disclosure during a preparation process.

### DETAILED DESCRIPTION

In order to make the purpose, technical solution and advantage of the embodiments of the present disclosure clearer, the technical solution of the embodiments of the present disclosure will be clearly and completely described below with reference to the drawings of the embodiments of the present disclosure. Obviously, the described embodiments are the portion of the embodiments of the present disclosure, but not all of them. Based on the described embodiments of the disclosure, all other embodiments obtained by those skilled in the art without creative labor are within the protection scope of the present disclosure.

Unless otherwise defined, the technical terms or scientific terms used in the disclosure shall have their common meanings as understood by those skilled in the art to which the disclosure belongs. The words “first”, “second” and the like used in the disclosure do not indicate any order, quantity or importance, but are only used to distinguish different components. The words “including”, “including” and the like mean that the elements or objects appearing before the words cover the listed elements or objects appearing after the words and their equivalents, without excluding other elements or objects. The words “connected”, “connecting” and the like are not limited to physical or mechanical connections, but may include electrical connections, whether direct or indirect. “Up”, “down”, “left” and “right” are only used to indicate the relative positional relationship. When the absolute position of the described object changes, the relative positional relationship may also change accordingly.

In a process of DNA sequencing by using a sequencing chip, in order to make a sequencing reaction of each DNA unit proceed independently and smoothly, for example, hundreds of millions of independent reaction separating units need to be formed in the sequencing chip, to support fixing of DNA molecules, implement high-throughput sequencing, and prevent detection crosstalk between adjacent units. In this regard, the structure of the independent reaction separating unit of the sequencing chip needs to be designed to meet the above-described requirements. In the process, how to form a structurally stable and high-throughput sequencing chip by low-cost means is a problem faced by those skilled in the art.

At least one embodiment of the present disclosure provides a detection chip and a preparation method thereof; the detection chip includes a base substrate and a detection layer, the detection layer is provided on the base substrate and includes a plurality of detection holes, wherein, at least some detection holes among the plurality of detection holes each have a hole wall provided with a hydrophilic layer, and a contact angle of the hydrophilic layer is within 20 degrees.

The above-described detection chip provided by the embodiment of the present disclosure may simply form a plurality of detection holes in the detection layer through a semiconductor preparation process to achieve the purpose of high throughput; the number of detection holes may reach hundreds of millions; at least some of the detection holes have a hole wall provided with a hydrophilic layer; and the hydrophilic layer has excellent hydrophilicity, so to-be-detected substances and detection reagents are more likely to accumulate in the detection holes, and crosstalk does not easily occur between adjacent detection holes, which, thus, may improve detection accuracy.

Hereinafter, the detection chip and the preparation method thereof provided by the embodiments of the present disclosure will be described in detail through several specific embodiments.

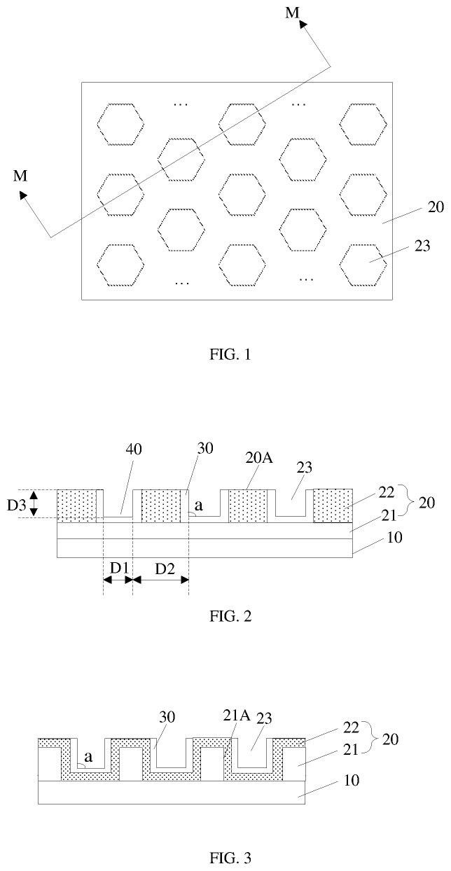

At least one embodiment of the present disclosure provides a detection chip; FIG. 1 shows a schematic plan view of the detection chip; and FIG. 2 shows a partial cross-sectional schematic diagram of the detection chip along an M-M line. As shown in FIG. 1 and FIG. 2, the detection chip includes a base substrate 10 and a detection layer 20; the detection layer 20 is provided on the base substrate 10 and includes a plurality of detection holes 23; at least some detection holes among the plurality of detection holes 23 each have a hole wall provided with a hydrophilic layer 30; and the hole wall at least includes a side wall of a detection hole 23, for example, the hole wall includes a side wall and a bottom surface of a detection hole 23 according to some embodiments.

For example, a contact angle of the hydrophilic layer 30 is within 20 degrees, for example, within 15 degrees, for example, within 10 degrees, for example, within 5 degrees, for example, 3 degrees or 4 degrees, etc. For example, in some embodiments, a thickness of the hydrophilic layer 30 may be 300 nm to 800 nm, for example, 400 nm, 500 nm, 600 nm, or 700 nm, etc.

In the embodiment of the present disclosure, the contact angle is a parameter of wettability of a liquid with respect to a surface of a solid material, and refers to an included angle from a solid-liquid interface through the interior of the liquid to a gas-liquid interface at a junction of three phases, that is, solid, liquid and gas; the smaller the included angle, the easier the liquid wets the solid, and the better the wettability are.

For example, in some embodiments, a contact angle of a surface 20A of the detection layer 20 that is away from the base substrate 10 is 80 degrees to 150 degrees, for example, 90 degrees to 150 degrees, for example, 120 degrees to 150 degrees, etc. Therefore, the surface 20A of the detection layer 20 between adjacent detection holes 23 is hydrophobic, and the interior of the detection hole 23 is hydrophilic, so that liquid in the detection hole 23, for example, a to-be-detected substance or a detection reagent, is more likely to accumulate within the detection hole 23, and is not easy to flow from one detection hole 23 into an adjacent detection hole 23 through the surface 20A of the detection layer 20, which, thus, may avoid detection crosstalk between adjacent detection holes 23 and improve detection accuracy.

For example, in some embodiments, slope angles a formed by side walls of at least some detection holes 23 among the plurality of detection holes 23 and a plate surface of the base substrate 10 are 85 degrees to 90 degrees, for example, 86 degrees to 89 degrees, for example, 87 degrees or 88 degrees, etc.

In the embodiment of the present disclosure, by setting the slope angles formed by side walls of at least some detection holes 23 and the plate surface of the base substrate 10 to 85 degrees to 90 degrees, crosstalk between adjacent detection holes may be effectively prevented; in addition, the structure of the detection hole 23 is more stable, which facilitates addition of a detection reagent. Through a test, when the slope angle formed by the side wall of the detection hole 23 and the plate surface of the base substrate 10 is less than 85 degrees, detection reagents in adjacent detection holes 23 are prone to crosstalk; when the slope angle formed by the side wall of the detection hole 23 and the plate surface of the base substrate 10 is greater than 90 degrees, it is difficult for the detection reagent to enter the detection hole 23, and during addition of the detection reagent, it is difficult to discharge gas in the detection hole 23, so it is difficult to ensure that there is sufficient detection reagent in the detection hole 23.

For example, in some embodiments, as shown in FIG. 2, a diameter D1 of the detection hole 23 may be 0.75 micrometers to 1.75 micrometers, for example, 0.9 micrometers, 1.0 micrometers, 1.2 micrometers or 1.5 micrometers, etc.; and a separation distance D2 between adjacent detection holes 23 may be 0.25 micrometers to 1.25 micrometers, for example, 0.5 micrometers, 1.0 micrometers, or 1.2 micrometers, etc.

In the embodiment of the present disclosure, when a size of a detection hole 23 is too large, a scanning speed will be slowed down when scanning to perform detection on the plurality of detection holes 23, thereby affecting detection efficiency; when a size of a detection hole 23 is too small, due to limited resolution of a detector, it is difficult to implement detection, resulting in missed detection. When the diameter D1 of the detection hole 23 and the separation distance D2 between adjacent detection holes 23 meet the above-described size requirements, requirements on the scanning speed and the detection resolution may be balanced.

For example, in some examples, if the diameter D1 of the detection hole 23 is 1.0 micrometer, and the separation distance D2 between adjacent detection holes 23 is also 1.0 micrometer, then in a detection chip with a size of 25 mm*65 mm, at least 400 million detection holes 23 may be formed, so that high throughput may be implemented.

For example, in some embodiments, as shown in FIG. 2, a depth D3 of the detection hole 23 may be 0.75 micrometers to 1.75 micrometers, for example, 0.9 micrometers, 1.0 micrometers, 1.2 micrometers, or 1.5 micrometers. Thus, the detection hole 23 may fully accommodate a detected substance and a detection reagent, and the detection reaction is completed.

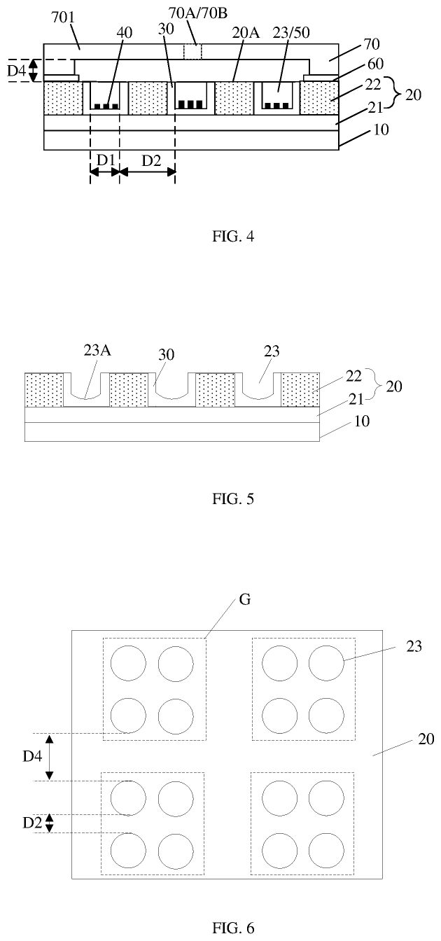

For example, in some embodiments, as shown in FIG. 4, the detection hole 23 is provided therein with an adapter primer 40; and the adapter primer 40 is connected to a surface of the hydrophilic layer 30 through a covalent bond. For example, the adapter primer 40 may be a segment of DNA, for connecting a to-be-detected DNA fragment.

In the embodiment of the present disclosure, by connecting the adapter primer 40 with the surface of the hydrophilic layer 30 through a covalent bond, the adapter primer 40 may be more firmly fixed within the detection hole 23 for subsequent reactions and detection steps. For example, in some examples, the covalent bond may be —CO—NH—.

For example, in some embodiments, as shown in FIG. 4, the detection chip may further include a cover layer 50; and the cover layer 50 is provided on a side of the adapter primer 40 that is away from the base substrate 10. The cover layer 50 may play a role of protecting the adapter primer 40, and is helpful to implement long-term preservation of the detection chip, which, for example, may be preserved for one year or even longer.

For example, FIG. 8 shows a validity test result of the adapter primer of the detection chip. As shown in FIG. 8, in a case where there is no cover layer 50 provided on the adapter primer 40, validity of the adapter primer 40 gradually decreases with time, and decreases to less than 50% within 12 months; and in a case where there is a cover layer 50 provided on the adapter primer 40, validity of the adapter primer 40 may be maintained basically 100% for a long time (at least within 12 months). It may be seen that the cover layer 50 may effectively protect the adapter primer 40 and greatly prolong preservation time of the detection chip.

For example, in some embodiments, a material of the cover layer 50 includes a water-soluble polymer such that the cover layer 50 may be removed through a simple water washing step, so as to expose the adapter primer 40.

For example, in some examples, the water-soluble polymer includes a copolymer of N-(5-azidoacetamidopentyl)acrylamide and acrylamide. In a preparation process, N-(5-azidoacetamidopentyl)acrylamide and acrylamide may be polymerized in relatively short reaction time by controlling a reaction temperature, so that preparation efficiency of the cover layer 50 may be improved; the copolymer of N-(5-azidoacetamidopentyl)acrylamide and acrylamide has good water solubility, may be removed through a simple water washing step, and ensures the adapter primer 40 not to be damaged.

For example, in some examples, a material of the detection layer 20 may include an inorganic material such as silicon nitride; and a material of the hydrophilic layer 30 may include an inorganic material such as silicon oxide. The silicon nitride material is easily treated to have better hydrophobicity, while silicon oxide is easily treated to have better hydrophilicity.

For example, in some embodiments, as shown in FIG. 2, the detection layer 20 may include a bonding material layer 21 and a hydrophobic material layer 22; the bonding material layer 21 has a flat structure; and the hydrophobic material layer 22 is provided on the bonding material layer 21, and has a plurality of detection holes 23. The hydrophobic material layer 22 may be firmly bonded to the base substrate 10 through the bonding material layer 21. For example, a hole formed in the hydrophobic material layer 22 may be a through hole, and at this time, the through hole exposes the bonding material layer 21, which is the case shown in FIG. 2; or, in other embodiments, the hole formed in the hydrophobic material layer 22 may also be a blind hole, and at this time, the blind hole will not expose the bonding material layer 21. For example, FIG. 3 shows another partial cross-sectional schematic diagram of the detection chip in FIG. 1 along the M-M line. In other embodiments, as shown in FIG. 3, the detection layer 23 may include a bonding material layer 21 and a hydrophobic material layer 22; the bonding material layer 21 includes a plurality of holes 21A; and the hydrophobic material layer 22 is provided on the bonding material layer 21, for example, provided on the bonding material layer 21 with a same thickness (i.e., a same thickness at respective positions), to form a plurality of detection holes 23 at positions of the plurality of holes 21A. For example, the hole formed by the bonding material layer 21 may be a through hole, and at this time, the through hole exposes the base substrate 10, that is, the case shown in FIG. 3; or, in other embodiments, the hole formed by the bonding material layer 21 may also be a blind hole, and at this time, the blind hole will not expose the base substrate 10.

For example, a material of the bonding material layer 21 includes Optical Clear (OC) adhesive; the optical clear adhesive has good bonding properties and light transmittance; and a material of the hydrophobic material layer 22 includes an inorganic material such as silicon nitride.

For example, in some embodiments, as shown in FIG. 5, a bottom face of the detection hole 23 that is close to the base substrate 10 includes an arc surface 23A that is concave toward a direction of the base substrate 10. The arc surface 23A is easier to be fabricated; and the adapter primer 40 formed on the arc surface 23A is easier to be connected.

For example, in some embodiments, a planar shape of the detection hole 23 may be a circle, an ellipse, or a polygon, for example, a square, a regular pentagon, or a regular hexagon (as shown in FIG. 1), etc. For example, when the planar shape of the detection hole 23 may be a square, the diameter of the detection hole 23 is a diagonal length of the square; when the planar shape of the detection hole 23 is a regular hexagon, the diameter of the detection hole 23 is a distance between opposite vertices or opposite sides of the detection hole 23; and other shapes are similar thereto.

For example, the plurality of detection holes 23 may be arranged in an array of a plurality of rows and a plurality of columns. For example, as shown in FIG. 1, when a planar shape of a detection hole 23 is a regular hexagon, the plurality of detection holes 23 may be arranged in a staggered manner, so that space of the base substrate 10 may be effectively utilized to have more detection holes 23 formed within a same area; or, in other embodiments, the plurality of detection holes 23 may also be arranged neatly in rows and columns.

For example, FIG. 6 shows a schematic plan view of another detection chip provided by an embodiment of the present disclosure; as shown in FIG. 6, in some embodiments, the plurality of detection holes 23 are divided into a plurality of groups of detection holes G (a portion shown by a dotted box); the plurality of groups of detection holes G are arranged in an array; each group of the plurality of groups of detection holes G includes a plurality of detection holes 23 arranged in an array; and a separation distance D4 between adjacent two groups of detection holes G among the plurality of groups of detection holes G is greater than a separation distance D2 between two adjacent detection holes 23 in each group of detection holes G. For example, the separation distance D4 between adjacent two groups of detection holes G may be a distance required for providing one detection hole 23, for example, which is approximately equal to D1+2D2.

For example, in the example shown in FIG. 6, a planar shape of a detection hole 23 is a circle; and the plurality of detection holes 23 are neatly arranged in rows and columns; and for example, in other embodiments, the plurality of detection holes 23 may be arranged in a staggered manner as shown in FIG. 1. For example, in the example shown in FIG. 6, a diameter of a detection hole 23 may be about 1 micrometer; a separation distance between adjacent detection holes 23 may be about 1 micrometer; and a separation distance between adjacent two groups of detection holes G may be about 3 micrometers.

For example, in some embodiments, as shown in FIG. 4, the detection chip may further include a cover plate 70; and the cover plate 70 is bonded by a binder 60 to a side of the detection layer 20 that is away from the base substrate 10, so as to implement encapsulation of the detection chip. For example, the cover plate 70 includes a sample inlet 70A and a sample outlet 70B; the sample inlet 70A may be used for adding a to-be-detected substance or a detection reagent; and the sample outlet 70B may be used for exhausting or discharging remaining to-be-detected substance or detection reagent after detection ends.

For example, the sample inlet 70A and the sample outlet 70B may be provided in an edge region of the cover plate 70. For example, in a case where a planar shape of the cover plate 70 is a rectangle, the sample inlet 70A and the sample outlet 70B may be respectively arranged in the periphery of the rectangle, for example, the sample inlet 70A and the sample outlet 70B may be respectively arranged on opposite sides of the rectangle; or arranged in two opposite corners of the rectangle.

For example, as shown in FIG. 4, the cover plate 70 includes a plate surface 70A which faces the base substrate 10, and at this time, the plate surface 70A is provided substantially parallel to the base substrate 10. For example, a distance D4 between the plate surface 70A and the detection layer 20 may be 50 micrometers to 100 micrometers, for example, 60 micrometers, 70 micrometers, 80 micrometers, or 90 micrometers, etc. Therefore, certain space may be formed between the plate surface 70A and the detection layer 20 to facilitate flow of gas or liquid, for example, to facilitate discharge of gas in the detection chip.

For example, in some embodiments, the cover plate 70 may be a glass cover plate or an organic cover plate (e.g., an acrylic cover plate, etc.). For example, the sample inlet 70A and the sample outlet 70B may be through holes in the plate surface 70A; or, in some embodiments, the sample inlet 70A and the sample outlet 70B may also have drainage structures correspondingly, so as to facilitate inflow or outflow of to-be-detected substances or detection reagents.

For example, a planar shape of the sample inlet 70A and the sample outlet 70B may be a regular shape such as a circle (as shown in the diagram), an ellipse, or a square, so as to quickly and efficiently implement operations such as sample addition or gas exhaust.

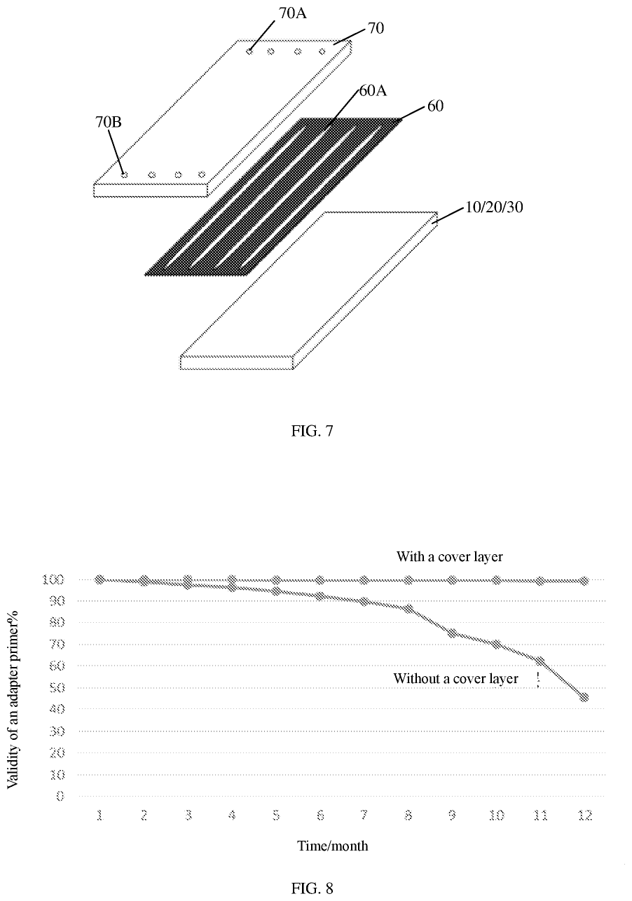

For example, FIG. 7 shows an exploded schematic diagram of the detection chip provided by the embodiment of the present disclosure; as shown in FIG. 7, in some embodiments, the binder 60 has a plurality of channel openings 60A; and each channel opening 60A may expose a plurality of groups of detection holes G, to form a plurality of groups of detection channels, so in a use process, the plurality of groups of detection channels may be respectively used for a plurality of groups of detection, thereby improving detection efficiency.

For example, in some embodiments, as shown in FIG. 7, each channel opening 60A is respectively provided with one sample inlet 70A and one sample outlet 70B correspondingly; and the sample inlet 70A and the sample outlet 70B are respectively arranged in two opposite sides of the channel opening 60A, and are provided in an edge region of the cover plate 70. Therefore, with respect to different detection channels, different to-be-detected substances or detection reagents may be respectively added or discharged through the sample inlet 70A and the sample outlet 70B, so that detections between the plurality of groups of detection channels are independent of each other.

For example, in other embodiments, the detection chip may also have only one detection channel, and at this time, the sample inlet 70A and the sample outlet 70B may be respectively arranged in middle positions of two opposite sides, so as to facilitate uniform diffusion of to-be-detected substances or detection reagents.

For example, in a process of sequencing detection by using the detection chip provided by the embodiment of the present disclosure, the cover layer 50 may be washed off with deionized water firstly to expose the adapter primer 40, then the to-be-detected DNA is connected to the adapter primer 40 in the plurality of detection holes 23, a detection reagent is added in the plurality of detection holes 23, so that the detection reagent may react with the to-be-detected DNA serially loaded in the plurality of detection holes 23, and emit fluorescence; and thereafter, an optical detector may be used to scan the plurality of detection holes 23, to detect colors of the fluorescence emitted in the plurality of detection holes 23, so as to obtain base types and a sequence thereof.

The above-described detection chip provided by the embodiment of the present disclosure may be prepared by using a semiconductor preparation process. As compared with a preparation method of a detection chip such as nano-imprinting and glass etching processes, the process flow of the semiconductor preparation process is fast and may effectively reduce the preparation cost.

At least one embodiment of the present disclosure further provides a preparation method of a detection chip; and the method includes: providing a base substrate, and forming a detection layer on the base substrate, the detection layer including a plurality of detection holes; wherein, at least some detection holes among the plurality of detection holes each have a hole wall provided with a hydrophilic layer; and a contact angle of the hydrophilic layer is within 20 degrees.

For example, in some embodiments, a contact angle of a surface of the detection layer that is away from the base substrate is 80 degrees to 150 degrees. For example, slope angles formed by side walls of at least some detection holes among the plurality of detection holes and a plate surface of the base substrate are 85 degrees to 90 degrees.

For example, in some embodiments, the base substrate may be a rigid substrate such as glass; and before use, the base substrate may be washed to maintain cleanliness thereof.

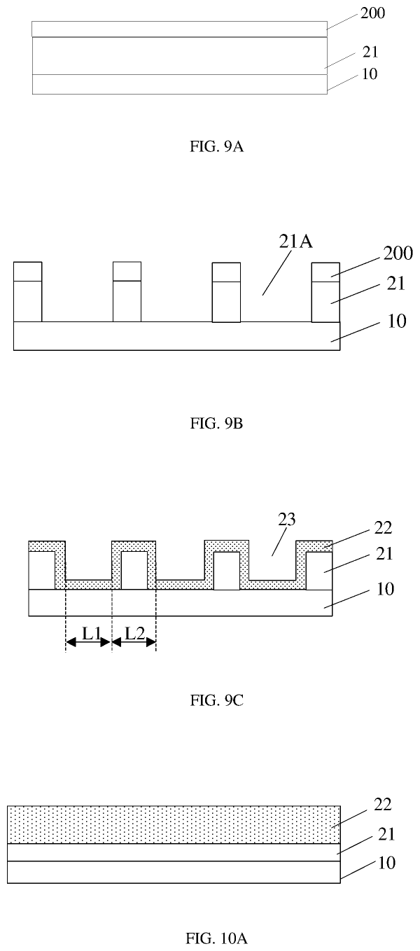

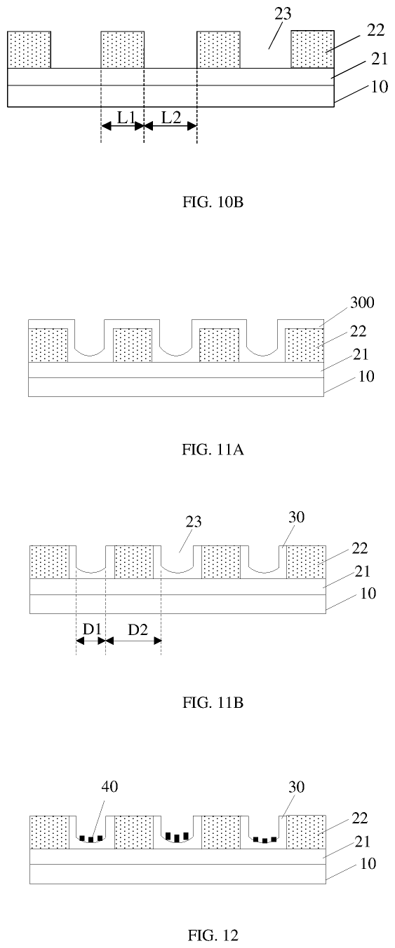

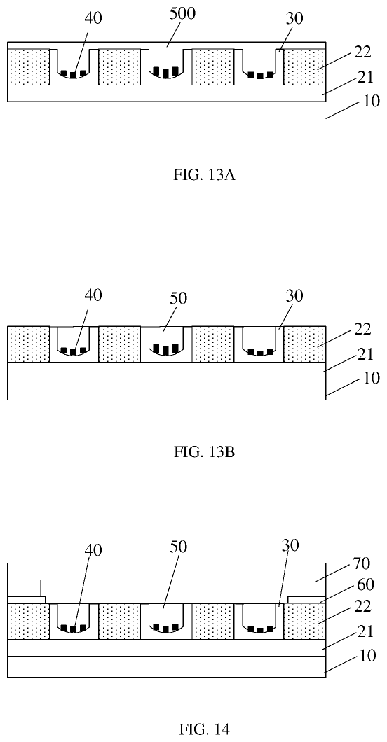

For example, in some embodiments, as shown in FIG. 9A to FIG. 9C, the forming a detection layer on the base substrate may include steps below.

First, as shown in FIG. 9A, a bonding material layer 21 and a metal material layer 200 are sequentially formed on the base substrate 10; then, the bonding material layer 21 and the metal material layer 200 are patterned, so that a plurality of holes 21A are formed in the bonding material layer 21 and the metal material layer 200; the plurality of holes 21A may be through holes (i.e., which expose the base substrate 10) or blind holes (i.e., which will not expose the base substrate 10), FIG. 9B shows the through holes as an example; thereafter, the metal material layer 200 is removed; and a hydrophobic material layer 22 is formed on a side of the bonding material layer 21 that is away from the base substrate 10, for example, the hydrophobic material layer 22 is formed with a same thickness, as shown in FIG. 9C. At this time, the detection layer 20 includes the bonding material layer 21 and the hydrophobic material layer 22; and a plurality of detection holes 23 are correspondingly formed at the positions of the plurality of holes 21A.

For example, the bonding material layer 21 may be made of Optical Clear (OC) adhesive, and may be formed on the base substrate by using a process of coating, for example, spin coating. The metal material layer 200 may be made of metal materials such as molybdenum, aluminum, copper, etc., and may be formed on the bonding material layer 21 by using a process of deposition or sputtering. The hydrophobic material layer 22 may be made of an inorganic material such as silicon nitride, and may be formed on the bonding material layer 21 by using a deposition process.

For example, the patterning the bonding material layer 21 and the metal material layer 200 may include processes such as formation, exposure, development, and etching, etc. of photoresist. Specific steps of the patterning process will not be limited in the embodiments of the present disclosure.

For example, in some examples, after the bonding material layer 21 and the metal material layer 200 are formed, photoresist is spin-coated on the metal material layer 200; and in the spin coating process, a rotation speed may be 250 rpm to 350 rpm, for example, 300 rpm; after spin coating, the photoresist is pre-baked, for example, for 1 minute to 3 minutes at a temperature of 80° C. to 100° C., for example, for 2 minutes at a temperature of 90° C.; then, spin coating may be repeated once; and the photoresist may be exposed with a mask; in the exposure process, an exposure intensity may be 100 mJ to 300 mJ, for example, 200 mJ, and a distance between the mask and the photoresist may be 50 micrometers to 150 micrometers, for example, 100 micrometers; exposure time may be 10 seconds to 20 seconds, for example, 15 seconds; then a development process is performed, for example, development is performed with a developer for 40 seconds to 50 seconds, for example, 45 seconds; and then, curing is performed at a temperature of 210° C. to 250° C., for example, 230° C. for 20 minutes to 40 minutes, for example, 30 minutes, thereby obtaining a photoresist pattern. Then, the photoresist pattern is used as a mask, to perform etching, for example, Inductive Coupled Plasma (ICP) etching, on the bonding material layer 21 and the metal material layer 200, to obtain the tiny hole array shown in FIG. 9B.

For example, the metal material layer 200 is used as an auxiliary forming layer, which is favorable for implementing precise patterning of the bonding material layer 21, so that the bonding material layer 21 is formed with a predetermined pattern. For example, a process such as etching may be used to remove the metal material layer 200.

For example, after the hydrophobic material layer 22 is formed at a side of the bonding material layer 21 that is away from the base substrate 10, an array of detection holes with a diameter L1 of 2 micrometers may be obtained; and at this time, a separation distance L2 between adjacent detection holes may be 0.5 micrometers, as shown in FIG. 9C.

For example, in other embodiments, as shown in FIG. 10A to FIG. 10B, the forming a detection layer on the base substrate may include steps below.

Firstly, as shown in FIG. 10A, a bonding material layer 21 and a hydrophobic material layer 22 are sequentially formed on the base substrate 100; and then, at least the hydrophobic material layer 22 is patterned to form a plurality of detection holes 23 at least in the hydrophobic material layer 22, as shown in FIG. 10B.

For example, the bonding material layer 21 may be made of Optical Clear (OC) adhesive, and may be formed on the base substrate by using a coating process. The hydrophobic material layer 22 may be made of an inorganic material such as silicon nitride, and may be formed on the bonding material layer 21 by using a deposition process.

For example, the patterning at least the hydrophobic material layer 22 may include processes such as formation, exposure, development, and etching, etc. of photoresist. For example, in some examples, after the bonding material layer 21 and the hydrophobic material layer 22 are formed, photoresist is spin-coated on the hydrophobic material layer 22; in the spin-coating process, a rotation speed may be 250 rpm to 350 rpm, for example, 300 rpm; after spin-coating, the photoresist is pre-baked, for example, for 1 minute to 3 minutes at a temperature of 80° ° C. to 100° C., for example, for 2 minutes at a temperature of 90° C.; then, spin-coating may be repeated once; the photoresist may be exposed with a mask; in the exposure process, an exposure intensity may be 100 mJ to 300 mJ, for example, 200 mJ, and a distance between the mask and the photoresist may be 50 micrometers to 150 micrometers, for example, 100 micrometers; exposure time may be 10 seconds to 20 seconds, for example, 15 seconds; then, a development process is performed, for example, development is performed with a developer for 40 seconds to 50 seconds, for example, 45 seconds; and then, curing is performed at a temperature of 210° C. to 250° C., for example, 230° C. for 20 minutes to 40 minutes, for example, 30 minutes, thereby obtaining a photoresist pattern. Then, the photoresist pattern is used as a mask, to perform etching, for example, Inductive Coupled Plasma (ICP) etching, on the hydrophobic material layer 22, to obtain an array of detection holes with a diameter L1 of 2 micrometers; and at this time, a separation distance L2 between adjacent detection holes may be 0.5 micrometers.

For example, in some embodiments, when patterning the hydrophobic material layer 22, the bonding material layer 21 may also be etched; at this time, a plurality of detection holes 23 are simultaneously formed in the bonding material layer 21 and the hydrophobic material layer 22.

For example, after the plurality of detection holes 23 are formed, the preparation method further includes: forming a hydrophilic layer on the hole walls of the detection holes 23.

For example, in some embodiments, as shown in FIG. 11A to FIG. 11B, the forming a hydrophilic layer on the hole walls of the detection hole 23 includes steps below.

Firstly, as shown in FIG. 11A, a hydrophilic material layer 300 is formed on a side of the hydrophobic material layer 22 that is away from the base substrate 10; then, the hydrophilic material layer 300 is patterned to reserve a portion of the hydrophilic material layer 300 that is inside a plurality of detection holes 23; a portion of the hydrophilic material layer 300 that is between adjacent detection holes 23 among the plurality of detection holes 23 is removed to form a hydrophilic layer 30, as shown in FIG. 11B.

For example, the hydrophilic material layer 300 may be made of an inorganic material such as silicon oxide; for example, the hydrophilic material layer 300 may be formed in a mode such as deposition; for example, a thickness of the hydrophilic material layer 300 may be 300 nm to 800 nm, for example, 700 nm; after the hydrophilic material layer 300 is formed, ICP etching or Reactive Ion Etching (RIE) may be performed on the hydrophilic material layer 300 to remove a portion of the hydrophilic material layer 300 that is located at an interval between adjacent detection holes, while reserving a portion inside the detection hole; at this time, the hydrophilic layer 30 is formed inside the detection hole 23; the diameter D1 of the detection hole 23 may be reduced to about 1 micrometer; and a separation distance D2 of adjacent detection holes may be enlarged to about 1 micrometer. In addition, slope angles formed by side walls of at least some detection holes 23 and the plate surface of the base substrate may be 87 degrees to 90 degrees, for example, 88 degrees, etc.

For example, in some embodiments, the bottom surface of the detection hole 23 is formed as an arc surface concave toward a direction of the base substrate 10. For example, during a sputtering process of the hydrophilic material layer 300, more material may be deposited in edge and corner positions of the detection hole 23, so as to form an arc surface and present a bowl-like structure.

For example, in some embodiments, the preparation method may further include: performing a first surface treatment on the hydrophilic layer 30 and the exposed hydrophobic material layer 22, so that a contact angle of the hydrophilic layer 30 is within 20 degrees, and a contact angle of the hydrophobic material layer 22 is 80 degrees to 150 degrees.

For example, in some examples, the first surface treatment may be a plasma treatment. For example, the hydrophilic layer 30 and the exposed hydrophobic material layer 22 are treated with plasma for a certain period of time, for example, 20 minutes to 40 minutes, for example, 30 minutes, to make the hydrophobic material layer 22 relatively hydrophobic, for example, have a contact angle of 80 degrees to 150 degrees, and make the hydrophilic layer 30 relatively hydrophilic, for example, have a contact angle within 20 degrees. For example, after the plasma treatment, the activation amount of terminal Si—OH of the hydrophilic layer 30 in a water solution environment is greatly increased.

For example, in some embodiments, the preparation method may further include: performing a second surface treatment on the hydrophilic layer 30, so that the surface of the hydrophilic layer 30 has a plurality of activating groups.

For example, in some embodiments, the second surface treatment may be performed with at least one of GPTMS, 12-mercaptododecanoic acid, and EDC and NHS.

For example, in some examples, the hydrophilic layer 30 is firstly treated with GPTMS; at this time, a methoxy group in a GPTMS molecule is firstly hydrolyzed with a water molecule to generate Si—OH; the Si—OH and the Si—OH on the surface of the hydrophilic layer 30 undergo a condensation reaction to form a Si—O—Si bond, which may be subsequently modified with 12-mercaptododecanoic acid; and an epoxy group and a sulfhydryl group are cross-linked, so that high-density —COOH is exposed on the surface of the hydrophilic layer 30, to implement super-hydrophilicity within the detection hole, for example, to have a contact angle less than 5 degrees, which may efficiently couple the adapter primer.

For example, in some embodiments, as shown in FIG. 12, the preparation method may further include: connecting adapter primers 40 within the plurality of detection holes 23. For example, the adapter primer 40 forms covalent bonds with a plurality of activating groups on the surface of the hydrophilic layer 30, to connect the adapter primer 40 to the surface of the hydrophilic layer 30 through the covalent bonds.

For example, in some examples, EDC and NHS are added to a detection hole 23, for example, EDC and NHS are added in a mass ratio of 1:1; then the adapter primer 40 is added; at this time, the adapter primer 40 may have a reaction with —COOH on the surface of the hydrophilic layer 30, so that the adapter primer 40 is connected within the detection hole 23 through a covalent bond, for example, the covalent bond is —CO—NH—; EDC and NHS are used as additives, which may improve a connection rate of the adapter primer 40 on the surface of the hydrophilic layer 30.

For example, in some embodiments, the preparation method may further include: forming a cover layer within the plurality of detection holes.

For example, in some embodiments, as shown in FIG. 13A to FIG. 13B, the forming a cover layer within the plurality of detection holes includes steps below.

Firstly, as shown in FIG. 13A, a cover material layer 500 is formed on a side of the detection layer 22 that is away from the base substrate 10; then, a portion of the cover material layer 500 that is located between the plurality of detection holes 23 is subjected to an ashing process or a polishing process, to remove the portion of the cover material layer 500 that is located between the plurality of detection holes 23, so that only a portion of the cover material layer 500 that is located inside the plurality of detection holes 23 remains, to form the cover layer 50, as shown in FIG. 13B.

For example, in some embodiments, the cover material layer 50 includes a copolymer of N-(5-azidoacetamidopentyl)acrylamide and acrylamide; and at this time, the forming a cover material layer 500 on a side of the detection layer that is away from the base substrate may include steps below.

Firstly, N-(5-azidoacetamidopentyl)acrylamide and acrylamide are pre-polymerized at a temperature of 40 degrees Celsius to 60 degrees Celsius, for example, 50 degrees Celsius, for 3 minutes to 8 minutes, for example, 5 minutes; then, a pre-polymerized product of N-(5-azidoacetamidopentyl)acrylamide and acrylamide is coated (e.g., spin-coated) on the side of the detection layer that is away from the base substrate and polymerized at a temperature of 30 degrees Celsius to 40 degrees Celsius, for example, at a temperature of 35 degrees Celsius, for 1 hour to 3 hours, for example, 2 hours, to complete polymerization of N-(5-azidoacetamidopentyl)acrylamide and acrylamide.

For example, the polymerized product of N-(5-azidoacetamidopentyl)acrylamide and acrylamide is water-soluble; so, before use of the detection chip, the polymerized product may be simply washed away with deionized water, so as to expose the adapter primer 40.

For example, the detection chip may be taken out every 1 month for validity test of the adapter primer, to verify validity of the adapter primer. For example, FIG. 10 shows a test result within 12 months. It may be seen that in a case where the cover layer 50 is provided on the adapter primer 40, validity of the adapter primer 40 may be maintained at substantially 100% validity for a long time (at least within 12 months).

For example, in some embodiments, as shown in FIG. 14, the preparation method may further include: coating a binder 60 on a side of the detection layer 22 that is away from the base substrate 10 and on at least the periphery of the detection layer 22; covering the cover plate 70 on a side of the binder 60 that is away from the base substrate 10; pre-baking the detection chip at a temperature of 90 degrees Celsius to 110 degrees Celsius, for example, 100 degrees Celsius, for 3 minutes to 6 minutes, for example, 5 minutes; then baking the detection chip at a temperature of 140 degrees Celsius to 160 degrees Celsius, for example, 150 degrees Celsius, for 8 minutes to 12 minutes, for example, 10 minutes, so as to firmly bond the cover plate 70 to the detection layer 22.

For example, a width for coating the binder 60 on the periphery of the detection layer 22 is 1.0 mm to 1.6 mm, for example, 1.2 mm; and after covering the cover plate 70, an overall thickness of the detection chip may be about 100 micrometers.

In the embodiment of the present disclosure, a high-throughput detection chip with good separation may be prepared through the above-described semiconductor preparation process, and the preparation process is simple, easy, and low in cost.

The following points need to be explained:
(1) The drawings of the embodiment of the present disclosure only involve the structures related to the embodiment of the present disclosure, and other structures can refer to the general design.
(2) For the sake of clarity, in the drawings used to describe the embodiments of the present disclosure, the thickness of a layers or a region is enlarged or reduced, that is, these drawings are not drawn to actual scale. It can be understood that when an element such as a layer, film, region or substrate is said to be located “above” or “below” another element, the element may be located “directly” above or below another element or intervening elements may exist.
(3) Without conflict, the embodiments of the present disclosure and the features in the embodiments can be combined with each other to obtain a new embodiment.

The above embodiments are only the specific embodiments of the present disclosure, but the protection scope of the present disclosure is not limited to this, and the protection scope of the present disclosure is the protection scope of the claims.

1. A detection chip, comprising:
a base substrate, and
a detection layer, provided on the base substrate and including a plurality of detection holes,
wherein, each of at least a portion of the plurality of detection holes has a hole wall provided with a hydrophilic layer, and a contact angle of the hydrophilic layer is within 20 degrees.

2. The detection chip according to claim 1, wherein, a contact angle of a surface of the detection layer that is away from the base substrate is 80 degrees to 150 degrees.

3. The detection chip according to claim 1, wherein, slope angles formed by side walls of at least a portion of the plurality of detection holes and a plate surface of the base substrate are 85 degrees to 90 degrees.

4. The detection chip according to claim 1, wherein, a diameter of the detection hole is 0.75 micrometers to 1.75 micrometers; and a separation distance between adjacent detection holes is 0.25 micrometers to 1.25 micrometers, wherein, a depth of the detection hole is 0.75 micrometers to 1.75 micrometers.

5. (canceled)

6. The detection chip according to claim 1, wherein, a bottom surface of the detection hole that is close to the base substrate comprises an arc surface that is concave toward a direction of the base substrate.

7. The detection chip according to claim 4, wherein, an adapter primer is disposed inside of the detection hole; and the adapter primer is connected to a surface of the hydrophilic layer through a covalent bond.

8. The detection chip according to claim 7, further comprising: a cover layer,
wherein, the cover layer is provided on a side of the adapter primer that is away from the base substrate, wherein, a material of the cover layer comprises a water-soluble polymer.

9. (canceled)

10. The detection chip according to claim 8, wherein, the water-soluble polymer comprises a copolymer of N-(5-azidoacetamidopentyl)acrylamide and acrylamide.

11. The detection chip according to claim 4, wherein, a material of the detection layer comprises silicon nitride; and a material of the hydrophilic layer comprises silicon oxide.

12. The detection chip according to claim 4, wherein, the detection layer comprises a binding material layer and a hydrophobic material layer; the binding material layer comprises a plurality of holes; and the hydrophobic material layer is provided on the binding material layer, to form a plurality of detection holes at positions of the plurality of holes, wherein, a material of the binding material layer comprises optical clear adhesive; and a material of the hydrophobic material layer comprises silicon nitride.

13. (canceled)

14. The detection chip according to claim 1, further comprising:
a cover plate, bonded by a binder to a side of the detection layer that is away from the base substrate, and comprising a sample inlet and a sample outlet,
wherein, the sample inlet and the sample outlet are arranged in an edge region of the cover plate, wherein, the cover plate comprises a plate surface opposite to the base substrate; and a distance between the plate surface and the detection layer is 50 micrometers to 100 micrometers.

15. (canceled)

16. The detection chip according to claim 1, wherein, the plurality of detection holes are divided into a plurality of groups of detection holes; the plurality of groups of detection holes are arranged in an array; and each of the plurality of groups of detection holes comprises a plurality of detection holes arranged in an array,
a separation distance between adjacent two groups of the plurality of groups of detection holes is greater than a separation distance between two adjacent detection holes of the plurality of detection holes in each of the plurality of groups of detection holes.

17. A preparation method of a detection chip, comprising:
providing a base substrate, and
forming a detection layer on the base substrate, the detection layer comprising a plurality of detection holes;
wherein, each of at least a portion of the plurality of detection holes has a hole wall provided with a hydrophilic layer; and a contact angle of the hydrophilic layer is within 20 degrees, wherein, the forming a detection layer on the base substrate, comprises:
sequentially forming a binding material layer and a metal material layer on the base substrate,
patterning the binding material layer and the metal material layer, so that a plurality of holes are formed in the binding material layer and the metal material layer,
removing the metal material layer, and
forming a hydrophobic material layer on a side of the binding material layer that is away from the base substrate,
wherein, the detection layer comprises the binding material layer and the hydrophobic material layer; and the plurality of detection holes are correspondingly formed at positions of the plurality of holes,
wherein, the forming a detection layer on the base substrate, comprises:
sequentially forming a binding material layer and a hydrophobic material layer on the base substrate; and
at least patterning the hydrophobic material layer, to form the plurality of detection holes at least in the hydrophobic material layer.

18. (canceled)

19. (canceled)

20. The preparation method according to claim 17, further comprising:
forming a hydrophilic layer on a hole wall of the detection hole,
wherein, the forming a hydrophilic layer on a hole wall of the detection hole, comprises:
forming a hydrophilic material layer on a side of the hydrophobic material layer that is away from the base substrate,
patterning the hydrophilic material layer, to retain a portion of the hydrophilic material layer that is inside the plurality of detection holes; and removing a portion of the hydrophilic material layer that is between adjacent detection holes of the plurality of detection holes.

21. (canceled)

22. The preparation method according to claim 20, further comprising:
performing a first surface treatment on the hydrophilic layer and the exposed hydrophobic material layer, so that a contact angle of the hydrophilic layer is within 20 degrees, and a contact angle of the hydrophobic material layer is 80 degrees to 150 degrees,
performing a second surface treatment on the hydrophilic layer, so that a surface of the hydrophilic layer has a plurality of activating groups,
wherein, the second surface treatment is performed with at least one of GPTMS, 12-mercaptododecanoic acid, and EDC:NHS.

23. (canceled)

24. The preparation method according to claim 22, wherein, the second surface treatment is performed with at least one of GPTMS, 12-mercaptododecanoic acid, and EDC:NHS.

25. The preparation method according to claim 20, further comprising:
connecting adapter primers within the plurality of detection holes,
forming a cover layer within the plurality of detection holes
wherein, the adapter primers form covalent bonds with a plurality of activating groups on the surface of the hydrophilic layer, to connect the adapter primers to the surface of the hydrophilic layer through the covalent bonds.

26. (canceled)

27. The preparation method according to claim 25, wherein, the forming a cover layer within the plurality of detection holes, comprises:
forming a cover material layer on a side of the detection layer that is away from the base substrate,
performing an ashing process or a polishing process on a portion of the cover material layer that is located between the plurality of detection holes, to remove the portion of the cover material layer that is located between the plurality of detection holes.

28. The preparation method according to claim 27, wherein, the cover material layer comprises a copolymer of N-(5-azidoacetamidopentyl)acrylamide and acrylamide; and the forming a cover material layer on a side of the detection layer that is away from the base substrate, comprises:
pre-polymerizing N-(5-azidoacetamidopentyl)acrylamide and acrylamide at a temperature of 40 degrees Celsius to 60 degrees Celsius for 3 minutes to 8 minutes; and
coating a pre-polymerized product of N-(5-azidoacetamidopentyl)acrylamide and acrylamide on the side of the detection layer that is away from the base substrate and polymerizing at a temperature of 30 degrees Celsius to 40 degrees Celsius for 1 hour to 3 hours.

29. The preparation method according to claim 17, further comprising:
coating a binder on a side of the detection layer that is away from the base substrate and on a periphery of the detection layer,
covering a cover plate on a side of the binder that is away from the base substrate,
pre-baking the detection chip at a temperature of 90 degrees Celsius to 110 degrees Celsius for 3 minutes to 6 minutes, and
baking the detection chip at a temperature of 140 degrees Celsius to 160 degrees Celsius for 8 minutes to 12 minutes.
